(19) United States
(12) Patent Application Publication
(10) Pub. No.: US 2012/0270890 A1
(43) Pub. Date: Oct. 25, 2012

(54) Methods of Pancreatic Beta Cell Regeneration

(76) Inventors: Cheng-Ho Chung, Taipei City (TW); Fred Levine, La Jolla, CA (US)

(21) Appl. No.: 13/452,192

(22) Filed: Apr. 20, 2012

Related U.S. Application Data
(60) Provisional application No. 61/478,144, filed on Apr. 22, 2011.

(51) Int. Cl. A61K 31/513; C12Q 1/68; A61P 3/10; G01N 21/64

(52) U.S. Cl. 514/274; 435/7.1; 435/6.17

(57) ABSTRACT

Disclosed are new methods for pancreatic β-cell regeneration and the methods for identifying adult pancreatic endocrine stem cells and the methods for identifying the existence of differentiation processes from adult pancreatic endocrine stem cells toward pancreatic β-cell fate and a new animal model for pancreatic β-cell regeneration. The present invention can be utilized in screening and development of new medicines and therapy protocols for diabetes.

### BACKGROUND OF THE INVENTION

1. Field of the Invention

The present invention relates to a method of cell regeneration, particularly to a method of pancreatic beta cell regeneration.

2. The Prior Arts

Diabetes mellitus is a group of diseases sharing the common phenotype of hyperglycemia due to absolute or relative insulin deficiency. Chronic hyperglycemia results in serious complications such as cardiovascular diseases, stroke, peripheral arterial diseases, retinopathy, neuropathy and nephropathy. In the past decade, the prevalence of diabetes has risen dramatically. The number of Americans with diagnosed diabetes in United States increased from 11 million in 2000, to 17.9 million in 2007. This increase is due primarily to an epidemic of obesity. Based on data from the World Health Organization, the number of people with diabetes worldwide in 2000 was around 171 million. However, it is estimated that the diabetic population will soar to over 366 million in 2030. The high prevalence rate and frequent complications render diabetes a leading cause of morbidity and mortality. In the foreseeable future, diabetes will be an enormous burden on health care systems and economies throughout the world.

Diabetes Mellitus (DM) is divided into 4 groups based on different etiologies and pathological processes. Type 1 DM develops as a result of autoimmune-mediated β cell destruction. Type 2 DM is characterized by insulin resistance, impaired insulin secretion and consequent relative insulin deficiency. In most patients, it is obesity associated, but there is increasing recognition of disease heterogeneity, with many genes and environmental stimuli contributing to disease pathogenesis. The third group of causes of diabetes is composed of a heterogeneous group of disorders, including monogenetic defects of β cell function, genetic defects in insulin action, diseases of the exocrine pancreas, endocrinopathies, drug and chemical induced diabetes, etc. Type 4 diabetes is gestational diabetes mellitus. The prevalence of each type of diabetes varies in different areas. The most common form of diabetes by far is type 2, accounting for about 90% of cases. Type 1 accounts for 5-10%, and other forms account for the remainder. Although they have different etiologies, type 1 and type 2 DM share the common feature of β cell deficit. The autoimmune attack on β cells in type 1 DM leads to almost complete loss of β cells and absolute insulin deficiency. While most type 2 DM patients do not suffer from autoimmunity, reduced β cell mass remains a significant feature of this form of diabetes. In humans, it has been reported that obese type 2 DM and impaired glucose tolerance patients had 63% and 40% deficits of β cell mass, respectively, compared to obese non-diabetic subjects. Impaired glucose tolerance is a prediabetic status whose blood glucose level, although not meeting criteria for diabetes, are too high to be considered normal. Please see the reference for its criteria. In individuals who develop type 2 diabetes without becoming obese, so-called “lean type 2 diabetes”, a 41% deficit in relative β cell mass was found compared to lean normal subjects. The mechanism underlying the loss of β cell mass in type 2 DM is thought to be increased β cell apoptosis caused by multiple aspects of the diabetic environment, including chronic supraphysiological glucose exposure (glucotoxicity), dyslipidemia (lipotoxicity) and increased proinflammatory cytokines. Hence, the decrease in β cell mass is an important pathological feature of both type 1 and type 2 diabetes and play a key role in insulin insufficiency and disease progression.

Since the loss of β cell mass is the major patho-physiological feature in diabetes, how to generate new β cells for patients becomes very important. The previous important approaches for β-cell replacement therapies and β-cell regeneration therapies include human islet transplantation (allo-transplantation), xeno-transplantation (for example, transplanting pig islets into humans), generating new β-cells from adult pancreatic ducts, generating new β-cells from embryonic stem (ES) cells and then transplanting these new β-cells into humans, generating new β-cells from pancreatic acinar cells and generating new β-cells from previous β-cells by triggering β-cell replication. The human islets for transplantation have to be harvested from cadaver and therefore, human islets transplantation is limited by the shortage of human islets. Besides, human islets transplantation is allo-transplantation. The transplanted islets will be attacked by autoimmunity. Transplanting non-human islets into humans is also limited by the immuno-rejection. In addition, use of non-human islets may also raise the concern of unknown infectious diseases.

People try to generate new β-cells in vivo from other sources. However, these approaches are still limited by the following obstacles. Adult pancreatic ductal cells have very limited ability to give rise to new β-cells, if any. It has been reported that new β-cells can be regenerated from pancreatic acinar cells by introducing 3 transcriptional factors. However, these new β-cells are single cells and they can't form new islets. It has been shown that the cell-cell contact between β-cells is critical for β-cell survival and for synergic insulin secretion. Besides, the applicant also has tested this method on human exocrine tissue in our laboratory. However, it was found that this method doesn't work for human exocrine tissue. Generating new β-cells from previous β-cells by inducing β-cell replication is very difficult because adult β-cell replication rate is extremely low, not to mention that there is no β-cell to begin with in type 1 diabetes. This age-dependent decline of β cell proliferation may result from the age-related increase of p16INK4a.

People also try to generate new insulin (+) from embryonic stem (ES) cells and then transplant these cells into humans. However, before its clinical application, several major obstacles still remain. First, tumorigenicity of ES cells is a concern. How to purify ES cell-derived insulin producing cells from other cells to obtain a homogenous cell population before transplantation is a critical issue. Second, allo-transplantation induces immune reactions, increasing the difficulty of using this new technology in patients, especially for type 2 diabetics. Although induced pluripotent stem cell technology has the potential to solve this problem, this will also increase the complexity and cost of treatment. Third, the efficiency of generating insulin producing cells from ES cells is still low. In addition, most of ES cell derived insulin (+) cells are immature and can't respond to glucose stimulation. Furthermore, how to scale up the whole manufacture process to industry level could be an issue. Finally, from patients' view, all new therapies need to offer a better outcome and lower risks when competing with existing treatments. Since diabetes became a chronic disease after the discovery of insulin, justifying the use of invasive transplantation procedures with possible complications is problematic. Currently, the best islet transplantation site is the hepatic portal system with possible complications such as bleeding, portal vein thrombosis and portal hypertension. A less invasive transplantation site might be needed.

The present invention provides great advantages to circumvent previous concerns. It has been proved by us that the identified adult pancreatic endocrine stem cells could rapidly generate a large amount of new β-cells in vivo. Therefore, there is no need for transplantation. There is no concern of immuno-rejection. Adult pancreatic endocrine stem cells are intact in type 1 diabetes and are increased in type 2 diabetes. In addition, adult pancreatic endocrine stem cells have the ability to self-renew and thus are able to provide a large pool of sources for β-cell regeneration. The present invention provides a simpler and safer strategy than other approaches to generate new β-cells. In summary, this approach is simpler and safer than other approaches and offers the highest possibility to be translated into clinical use.

In comparison with Fabrizio et al (Nature. Apr. 22, 2010; 464(7292):1149-54), they demonstrated that adult α-cells are β-cells' progenitors. However, we discovered that adult alpha cells could give rise to new beta cells independently from their research. In addition, they didn't find out that α-cells have stem cell characters which are self-renewal and differentiation. These are the sentences quoted from their paper (page 1150): “Islets became prominently composed of a cells, yet α-cell proliferation and mass remained unchanged during the entire analysis period (up to 10 months; Supplementary FIG. 5d, e)”. This proved that they didn't discover that adult α-cells have self-renewal ability. Therefore, they didn't discover the fact that adult pancreatic α-cells are stem cells.

### SUMMARY OF THE INVENTION

In diabetic patients, β-cell deficit is a major pathological feature and plays a key role in disease progression. β-cell regeneration to replace the cells lost in diabetes is a promising approach to develop anti-diabetes therapies. However, in the past, because the adult pancreatic endocrine stem cells were unknown, many approaches to β-cell regeneration have failed.

The present invention provides methods of identifying adult pancreatic endocrine stem cells and methods of identifying the existence of differentiation processes from adult pancreatic endocrine stem cells toward pancreatic β cell fate. Diabetes is a disease with high prevalence rate and serious complications and is an enormous burden on health care systems and economies throughout the world. In diabetic patients, β cell deficit is a major pathological feature and plays a key role in disease progression. β cell regeneration to replace the cells lost in diabetes is a promising approach to develop anti-diabetes therapies. However, in the past, because the adult pancreatic endocrine stem cells were unknown, many approaches to β-cell regeneration have failed. Herein, we identified adult pancreatic endocrine stem cells. The identified cells are shown to possess unique properties of stem cells, including self-renewal and differentiation into pancreatic β-cells. In addition, the present invention demonstrates that the cells can rapidly generate a large amount of β-cells. The adult pancreatic endocrine stem cells have the phenotype of “glucagon (+)”, “glucagon (+) and PDX1 (−)”, “glucagon (+) and NKX6.1 (−)”, or “glucagon (+) and PDX1 (−) and NKX6.1 (−)”. The presence of “glucagon (+) cells”, “glucagon (+) and PDX1 (−) cells”, or “glucagon (+) and PDX1 (−) and NKX6.1 (−) cells” indicates the presence of adult pancreatic endocrine stem cells and allows the definition of adult pancreatic endocrine stem cell domains in the adult pancreas. The present invention provides a way that therapies for beta cell regeneration and anti-diabetes can be directed, both generally and specifically, toward adult pancreatic endocrine stem cells.

The present invention also provides a method to identify the existence of differentiation processes from adult pancreatic endocrine stem cells toward pancreatic β-cell fate. The method comprises: (a) providing a sample of pancreatic tissue or pancreatic cells; and (b) detecting glucagon, pancreatic and duodenal homeobox 1(PDX1), NK6 homebox 1(NKX6.1), insulin and v-maf musculoaponeurotic fibrosarcoma oncogene homolog B(MafB) expression of the sample; wherein the existence of cells having the phenotype of expressing at least two markers selected from the group consisting of glucagon, PDX1, NKX6.1, insulin and MafB, indicates the existence of differentiation processes from adult pancreatic endocrine stem cells toward pancreatic beta cell fate.

Preferably, the existence of cells having the phenotype of expressing glucagon and NKX6.1 or cells having the phenotype of expressing glucagon and PDX1 or cells having the phenotype of expressing glucagon, PDX1 and MafB or cells having the phenotype of expressing insulin, glucagon and MafB or cells having the phenotype of expressing insulin, glucagon and PDX1 or cells having the phenotype of expressing insulin, glucagon and NKX6.1 or cells having the phenotype of expressing insulin and glucagon, indicates the existence of differentiation processes from adult pancreatic endocrine stem cells toward pancreatic beta cell fate.

The present invention also provides a method for generating new pancreatic beta cells from adult pancreatic endocrine stem cells which are pancreatic alpha cells in vivo in animals. The method comprises: (a) eliminating pre-existing pancreatic beta cells; and (b) inducing apoptosis of acinar cells. Preferably, the elimination of pre-existing pancreatic beta cells in step (a) is by administration of alloxan or any methods to eliminate pre-existing pancreatic beta cells, and inducing apoptosis of acinar cells in step (a) is by performing a pancreatic ductal ligation or methods to induce apoptosis of pancreatic acinar cells.

The present invention also provides a new animal model to generate new pancreatic beta cells from adult pancreatic endocrine stem cells which are alpha cells in vivo in animals. This new animal model is to perform pancreatic ductal ligation (PDL) and alloxan administration on animals. This new animal model can be utilized in screening and development of new factors or molecules or compounds or medicines or therapy protocols for diabetes. This new animal model also can be used for testing beta cell regeneration therapy and anti-diabetes therapy.

The present invention has many immediate and future applications. The present invention could be used immediately. For example but not limited to, the present invention could be used to identify adult pancreatic endocrine stem cells. Adult pancreatic endocrine stem cells may be used, for an example but not limited to, as targets for the discovery of factors or molecules or compounds that can affect their self-renewal and differentiation into β-cells. The present invention is also useful for experimental evaluation. The methods to identify the presence of differentiation from adult pancreatic endocrine stem cells toward pancreatic β-cell fate could be used immediately for drug screening to find compounds or factors or molecules which can trigger adult pancreatic endocrine stem cells to differentiate toward pancreatic β-cell fate and eventually generating new β-cells. The cells and the methods are also useful in identifying specific genes or proteins or pathways or any other factors which could affect the self-renewal or differentiation ability of adult pancreatic endocrine stem cells. In the future, the present invention is useful in developing and testing new anti-diabetes therapies and new β-cells regeneration therapy and is used as a model for identification of new therapeutic targets for beta cell regeneration and anti-diabetes therapies.

In summary, the present invention provides useful methods for identifying adult pancreatic endocrine stem cells and the methods for identifying the existence of differentiation processes from adult pancreatic endocrine stem cells toward pancreatic β-cell fate. Adult pancreatic stem cells are useful as targets for the discovery of factors or molecules or compounds that can affect their ability to generate new β-cells and to self-renew. The present invention also provides useful methods for testing beta cell regeneration therapy and anti-diabetes therapy; for the development of drugs or compounds or factors to generate new β-cells or create new anti-diabetes therapies. The invention could also be used as a model for identification of new therapeutic targets for beta cell regeneration and anti-diabetes therapies.

### BRIEF DESCRIPTION OF THE DRAWINGS

The present invention will be apparent to those skilled in the art by reading the following detailed description of preferred embodiments thereof, with reference to the attached drawings, in which:

FIG. 1(A)˜(J). Alloxan plus PDT induces rapid and robust β-cell neogenesis in the adult mouse pancreas. Representative sections from the pancreas of a normal control (A), a mouse 14 days after alloxan injection (C), and from the distal pancreas 3 (B), 7 (D), and 14 (E) days following alloxan and PDL. Sections were immunostained with antibodies against insulin (red) and glucagon (green). Nuclei were visualized with DAPI (blue). Scale bar=50 lm. Alloxan injection eliminated 99% of pre-existing β-cells by 3 days after injection,while α-cells remained intact (compare [A] with [B], quantified in [F]). There was no change in the β-cell mass between 3 and 14 days after alloxan injection ([B, C], quantification in [F]). α-cell hyperplasia and numerous insulin (+) cells appear at day 7 post PDL+A (D). Large islets mainly composed of β-cells are seen 14 days post PDL+A (E). Quantification of β-cell mass (F). β-cell replication does not contribute significantly to β-cell regeneration in the PDL+A model (G-J), The replication of insulin (+) cells (green) at 3 days (G), 7 days (H), and 14 days (I) after PDL+A was analyzed by immunohistochemistry for KO67 (red). β-cell replication was low at all time points (quantification in [J]). Arrows indicate representative cells positive for KI67. Scale bars=10 um. Data are presented as mean±SD, n=3 animals; *, p<0.05. Abbreviations: DAPI, DAPI (40,6-diamidino-2-phenylindole); PDL, pancreatic duct ligation.

FIG. 1(K)˜(L), Transitional endocrine cells in human pancreas, Representative pancreatic sections from an adult human patient without pancreatitis are shown in (K), and from an adult human patient with pancreatitis in (L). Endocrine cells in the adult human pancreas do not coexpress insulin (red) and glucagon (green) (K), but such double positive cells were found in patients with pancreatitis (L, indicated by arrows). Scale bar=20 um (K and L).

FIG. 2. Neogenic insulin (+) cells are initially immature but mature over time. Mouse pancreas from normal adult ([A] and [B], left panel), 7 days after PDL+A ([A] and [B], middle panel), and 14 days after PDL+A ([A] and [B], right panel) were examined. Costaining with insulin (green), MafB (red), and DAPI (blue) in (A) and insulin (green), MafA (red), and DAPI (blue) in (B) are shown. As expected, mature normal adult β-cells expressed MafA, not MafB (left panel in [A] and [B]). At day 7 after PDL+A, numerous insulin (+) cells expressed MafB (middle panel in [A]) and only a small number of insulin (+) cells expressed MafA (middle panel in [B]). However, at day 14 post PDL+A, numerous insulin (+) cells expressed MafA (right panel in [B]) and only a small number of insulin (+) cells expressed MafB (right panel in [A]), consistent with β-cell maturation over time. Scale bar=10 um (A, B). Quantification of MafB (C) and MafA (D) expression in insulin (+) cells. Data are presented as mean±SD, n=3; *, p<0.05. **, p<0.01. Abbreviations: DAPI, DAPI (40,6-diamidino-2-phenylindole); PDL, pancreatic duct ligation.

FIG. 3. Endocrine neogenesis from ducts does not change with PDL+A compared with PDL alone. Representative pancreatic sections 7 days after PDL+A (A, C) and 7 days after PDL alone (B, D). Sections were immunostained with antibodies against insulin (red in [A] and [B]), glucagon (red in [C] and [D]), and pancytokeratin (PanCK, green). DAPI (blue) was used to visualize nuclei. Scale bar=50 lm. Islets are outlined with white dashed lines. Insulin (+) and glucagon (+) cells in the ducts are rare in both PDL+A and PDL alone models (A-D). The arrow in (B) indicated an insulin (+) cell in the hyperplastic duct. Quantification of the frequency of ducts with insulin (+) or glucagon (+) cells (E). Quantification of the number of endocrine cells in each duct containing endocrine cells (F). In (E) and (F), over 5,000 ducts were counted in the insulin group. Over 4,000 ducts were counted in the glucagon group. Data are presented as mean±SD, n=3 animals. Student's t-test results are shown. Abbreviations: DAPI, (40,6-diamidino-2-phenylindole); n.s., not significant; PanCK, pancytokeratin; PDL, pancreatic duct ligation.

FIG. 4. Neogenic β-cells induced by PDL plus alloxan arise from pre-existing α-cells. Photomicrographs of adult mouse pancreas seven (A) and 14 (B, C) days after PDL+A. The boxed regions in (B) are shown at high power in (C). Insulin (red) and glucagon (green) staining show numerous cells coexpressing both markers. Colocalization of insulin and glucagon (white) was determined by the colocalization threshold algorithm in Image J (right panel). Scale bar=10 um (A, C); 50 um (B). Quantification of insulin (+) cells coexpressing Data are presented as Lean±SD, n=3 animals. Abbreviations: DAN, (40,6-diamidino-2-phenylindole); PDL, pancreatic duct ligation.

FIG. 5. PDL plus alloxan induces α-cell proliferation and PDX1 expression. PDL+A induced α-cell proliferation. Seven days after PDL+A, the endocrine compartment of the ligated part of the pancreas was mainly composed of hyperplastic α-cells (green) (A). Most α-cells in the normal adult mouse pancreas did not express Ki67 ([B], left panel). However, PDL+A induced a large increase in Ki67 expression in α-cells (arrows in [B], right panel). Quantification of Ki67 and PDX1 expression in α-cells (C). Data are presented as mean±SD, n=3 animals. *, p<0.05. Scale bar=50 um (A); 10 um (B). Abbreviations: DAPI, (40,6-diamidino-2-phenylindole); PDL, pancreatic duct ligation.

FIG. 6. Characterization of endocrine cells following PDL+A. (A, C, E, G): Endocrine cells from normal adult pancreas. (B, D, F, H, I, J): Endocrine cells are from 7 days post PDL+A. As expected, glucagon (+) cells in the noadult pancreas did not express PDX1 (A). PDL+A induced PDX1 expression in glucagon (+) cells ([B], representative cells indicated by arrows). As expected, glucagon (+) cells in adult pancreas did not express Nkx6.1 (C). PDL+A induced Nkx6.1 expression in glucagon(+) cells ([D], representative cells indicated by arrows). As expected, glucagon (+) cells in the normal adult mouse pancreas expressed MafB, but not PDX1 (E). PDL+A induced MafB (red) and PDX1 (green) coexpression in glucagon (+) cells ([F], yellow nuclei indicated by arrows). As expected, mature β-cells in the normal adult pancreas did not express MafB (G). PDL+A induced cells coexpressing insulin (green), glucagon (red), and MafB (blue; [H], a representative cell indicated by the arrow). Cells (+) only for insulin did not express MafB (arrowheads). Cells coexpressing insulin (red) and glucagon (green; yellow cells indicated by arrows) also expressed nuclear PDXI (I) and Nkx6.1 (J), Scale bar=10 um (A-J). Abbreviation: DAPI, (40,6-diamidino-2-phenylindole).

FIG. 7. Replication is not required for α- to β-cell conversion (A, B). Continuous labeling with BrdU for 12 days (day 3-14 after PDL+A) (A). Cells (yellow) coexpressing insulin (red) and cagon (green) develop with (long arrow) or without (short arrow) having replicated as indicated by BrdU incorporation. The lack of BrdU incorporation in some α-cells (denoted by an arrowhead) in this islet is expected for α-cells that were part of a pre-existing islet. Immunohistochemistry, insulin (red), glucagon (green), and KI67 (blue), 7 days after PDL+A reveals cells coexpressing insulin and glucagon (yellow cells) that are negative for Ki67 (B). Arrowheads denotes KI67 (+) and glucagon (+) cells. β-cell neogenesis in mature mice (C). Pancreatic sections from 7-month-old mice 14 days after PDL+A. The boxed area is shown at high power on the right. Transitional cells coexpressing insulin and glucagon (arrows) still formed in mature mice. Scale bar=10 um ([A, B] and right panel of [C]); 50 um (left panel of [C]). Abbreviations; BrdU, 5-bromodeoxyuridine; DAPI, (40,6-diamidino-2-phenylindole).

FIG. 8. Numerous β-cells are formed after PDL plus alloxan. Representative sections from additional pancreases 14 days after PDL+A (A to C) and from 14 days after alloxan alone (D) were stained with antibodies against insulin (red), glucagon (green) and DAPI (blue). The boxed area in A is shown at high power in C. Scale bar, 50 um (A, B and D); 10 um (C).

FIG. 9. Replicating mature β-cells do not express MafB. A pancreatic section from a 4-week-old mouse was immunostained with insulin (blue), Ki67 (green), MafB (red) and DAPI (white). The arrow points to an insulin (+)/Ki67 (+) cell that was negative for MafB. Scale bar, 10 um.

FIG. 10. Most neogenic β-cells did not originate from o-cells. Serial sections from pancreases 7 (A) and 14 (B) days after PDL+A were immunostained with somatostatin(green), insulin (red) and DAPI (blue), C localization of insulin and somatostation (white) was determined by the colocalization threshold algorithm in Image J. Most insulin (+) cells did not coexpress somatostatin. Scale bar, 10 um. (C) Quantification of insulin (+) cells coexpressing somatostatin. Data are presented as mean±s.d., n=3 animals.

FIG. 11. Neogenic β-cells originate from mature α-cells in the model of PDL+A. Additional sections from pancreases harvested 14 days after PDL+A (A to C). Sections were immunostained with insulin (red), glucagon (green) and DAPI (blue). Numerous cells co-expressed insulin and glucagon. The colocalization patterns (white) are shown in lower right panels of A and B. The boxed region in A is shown high power in B. Orthogonal views from stacks of confocal Z series of the islet in B, demonstrating colocalization of insulin and glucagon (C).

FIG. 12. Model of α- to β-cell conversion. After PDL+A, mature α-cells have at least 3 different choices in response to environmental cues. They can replicate, leading to α-cell hyperplasia. α-cells can also convert directly into β-cells, with no intervening cell division. Finally, α-cells can proliferate first and then convert into β-cells. Black arrows indicate cell replication. Orange arrows denote the process of α- to β-cell conversion.

FIG. 13. β-cell Neogenesis in mature mice. 7-month-old mice underwent PDL+A and pancreases were harvested 14 days later. Sections were immunostained with insulin (red), glucagon (green) and DAPI (blue). The boxed area in A is shown at high power in B. Single channels of B are presented (C to E). The arrow indicates a representative cell coexpressing insulin and glucagon. Scale bar, 50 um (A); 10 um (B to E).

FIG. 14, Insulin (+) cells regenerated 14 days after PDL+A also express C-peptide. Representative sections from pancreases 14 days after PDL+A were stained with insulin (red), C-peptide (green) and DAPI (blue). All insulin (+) cells express C-peptide (yellow cells in Merge panel). Scale bar, 50 um.

FIG. 15, α-cells perform self-renewal after PDL (pancreatic ductal ligation) plus alloxan. A representative section from pancreases after continuous labeling with BrdU for 12 days (day 3-14 after PDL+alloxan) was stained with BrdU(red), glucagon (green) and DAPI (blue).Cells expressing glucagon (green) perform cell replication as indicated by BrdU incorporation (denoted by arrows).

FIG. 16. Numerous β-cells are formed after PDL plus alloxan. Representative sections from additional pancreases 14 days after PDL+A (A to C) and from 14 days after alloxanalone (D) were stained with antibodies against insulin (red), glucagon (green) and DAPI (blue). The boxed area in A is shown at high power in C. Scale bar, 50 um (A, B and D); 10 um (C).

FIG. 17. Replicating mature β-cells do not express MafB. A pancreatic section from a 4-week-old mouse was immunostained with insulin (blue), Ki67 (green), MafB(red) and DAPI (white). The arrow points to an insulin (+)/Ki67 (+) cell that was negative for MafB. Scale bar, 10 um.

FIG. 18. Most neogenicβ-cells did not originate from δ-cells. Serial sections from pancreases 7 (A) and 14 (B) days after PDL+A were immunostained with somatostatin(green), insulin (red) and DAPI (blue). Colocalization of insulin and somatostation(white) was determined by the colocalization threshold algorithm in Image J. Most insulin (+) cells did not coexpress somatostatin. Scale bar, 10 um. (C) Quantification of insulin (+) cells coexpressing somatostatin. Data are presented as mean+s.d., n=3 animals.

FIG. 19. Neogenicβ cells originate from mature a cells in the model of PDL+A. Additional sections from pancreases harvested 14 days after PDL+A (A to C). Sections were immunostained with insulin (red), glucagon (green) and DAPI (blue). Numerous cells co-expressed insulin and glucagon. The colocalization patterns (white) are shown in lower right panels of A and B. The boxed region in A is shown at high power in B. Orthogonal views from stacks of confocal Z series of the islet in B, demonstrating colocalization of insulin and glucagon (C).

FIG. 20. β-cell Neogenesisin mature mice. 7-month-old mice underwent PDL+A and pancreases were harvested 14 days later. Sections were immunostained with insulin (red), glucagon (green) and DAPI (blue). The boxed area in A is shown at high power in B. Single channels of B are presented (C to E). The arrow indicates a representative cell coexpressing insulin and glucagon. Scale bar, 50 um (A); 10 um (B to E).

FIG. 21. Insulin (+) cells regenerated 14 days after PDL+A also express C-peptide. Representative sections from pancreases 14 days after PDL+A were stained with insulin (red), C-peptide (green) and DAPI (blue). All insulin (+) cells express C-peptide (yellow cells in Merge panel). Scale bar, 50 um.

### DETAILED DESCRIPTION OF THE PREFERRED EMBODIMENTS

Six- to ten-week old C57/B6 or ICR (Institute of Cancer Research) mice (Harlan Sprague Dawley, Inc.; Placentia, Calif., USA) were injected intravenously with Alloxan (Sigma Aldrich, St. Louis, Mo., USA) at 90 mg/kg. PDL was conducted essentially as described with the following minor modifications. Approximately 50% of the pancreas (the head) remained proximal to the ligation. For the mice receiving PDL plus A, PDL was performed 30 minutes after alloxan injection. Only mice with a blood sugar greater than 500 mg/dl (checked 48 hours after alloxan injection) were selected for experiments. Those mice were injected subcutaneously once daily with insulin glargine (Sanofi-Aventis, Paris, France).

The initial insulin dose was 1 unit per mouse (around 25 mg in weight). If hyperglycemia was not controlled at that dose, it was raised gradually until diabetes was controlled. Even with careful management of diabetes, the mortality following PDL plus alloxan was high, around 50%.

Immunohistochemical Staining—Tissue was fixed in 4% formaldehyde for 6 hours at 4° C., washed in PBS (Phosphate buffered saline), followed by overnight in 30% sucrose at 4° C., then embedded in OCT (Optimal Cutting Temperature) compound and frozen at 80° C. Cryosections of 5 um thickness were incubated with antisera specific for insulin (1/200, guinea pig, USBIO, Swampscott, Mass., USA), insulin (1/200, rabbit, Santa Cruz Biotechnology, Santa Cruz, Calif., USA), glucagon (1/2,000, mouse, Sigma-Aldrich, St. Louis, Mo., USA), glucagon (1/50, rabbit, Abcam, Cambridge, Mass., USA), cytokeratin (Wide Spectrum Screening; 1/500, rabbit, DakoCytomation, Glostrup, Denmark), somatostatin (1/200, goat, Santa Cruz Biotechnology, Santa Cruz, Calif., USA), KI67 (1/50, mouse, BD Pharmingen, San Diego, Calif., USA), MafA (1/100, rabbit, Bethyl Laboratories, Montgomery, Tex., USA), MafB (1/100, rabbit, Bethyl Laboratories, Montgomery, Tex., USA), PDX1 (1/2,000, goat, Abcam, Cambridge, Mass., USA), Nkx6.1 (1/1,000, mouse, Beta Cell Biology Consortium, Nashville, Tenn., USA), 5-bromodeoxyuridine (BrdU; mouse, GE healthcare, Piscataway, N.J., USA), and C-peptide (1/100, rabbit, Cell Signaling, Danvers, Mass., USA). Secondary antibodies for detection of guinea pig, rabbit, goat, or mouse antibodies were labeled with: Alexa Fluor 488 (Invitrogen, Carlsbad, Calif., USA), Rhodamine Red (Jackson ImmunoResearch Laboratories, West Grove, Pa., USA), Cy5 (Jackson ImmunoResearch Laboratories, West Grove, Pa., USA). Nuclei were visualized with DAPI (40,6-diamidino-2-phenylindole) (Sigma Aldrich, St. Louis, Mo., USA).

Quantitation of β-cell Mass—β-cell mass was calculated as the relative β-cell area multiplied by pancreatic weight. For the quantitative analysis of insulin (+) cell area, we studied sections spaced 100 lm apart from each other from the tail of the pancreas per mouse. These sections were incubated with antisera to insulin (1:200, rabbit, Santa Cruz Biotechnology, Santa Cruz, Calif., USA) and the bound antibody was visualized by DAB (3,30-Diaminobenzidine) (Vector Laboratories, Burlingame, Calif., USA). The nuclei were stained by Hematoxylin (Surgipath, Richmond, Ill., USA). All slides were scanned and analyzed by using the Aperio ScanScope XT system (version 10, Aperio Technologies, Vista, Calif.).

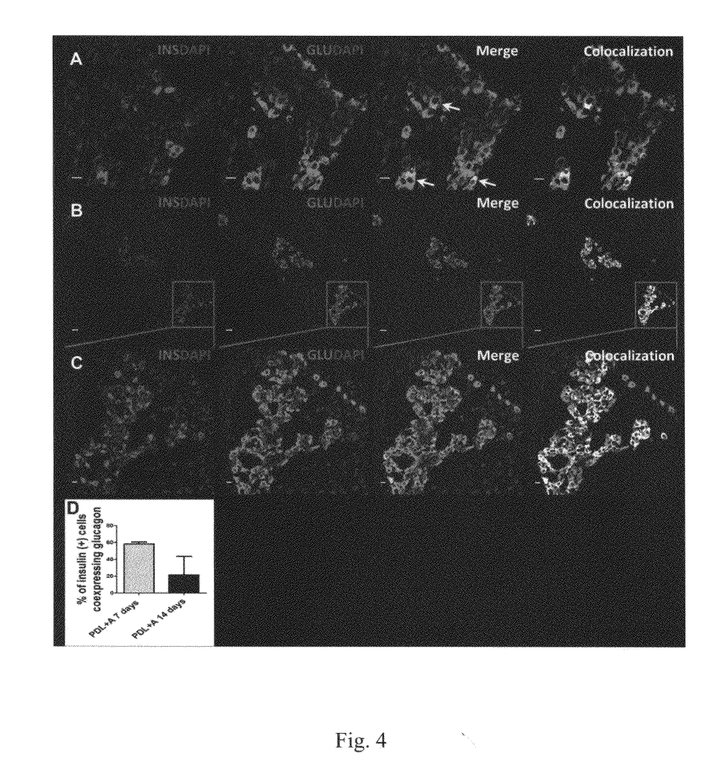

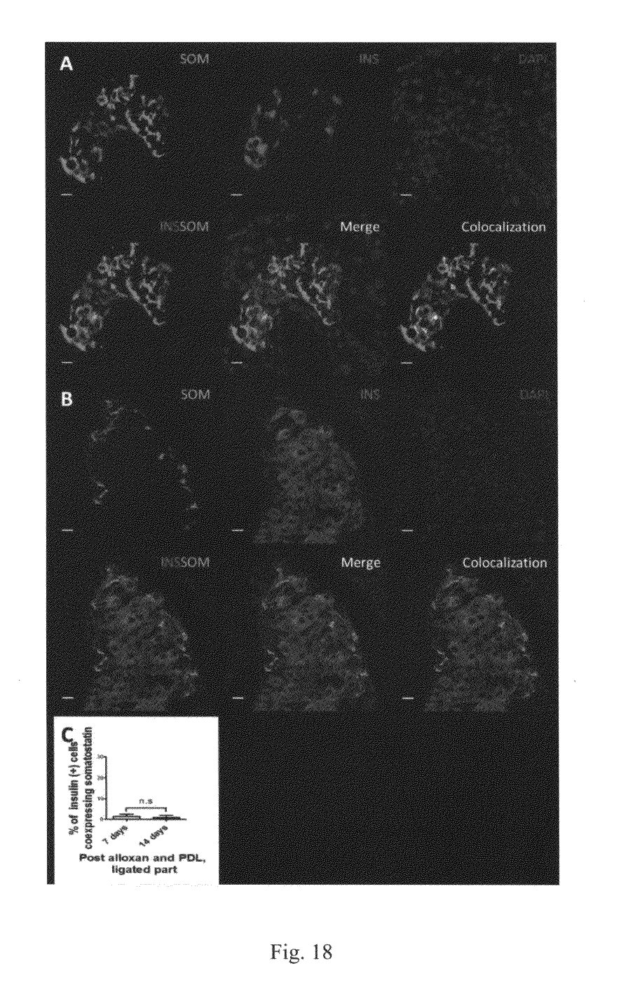

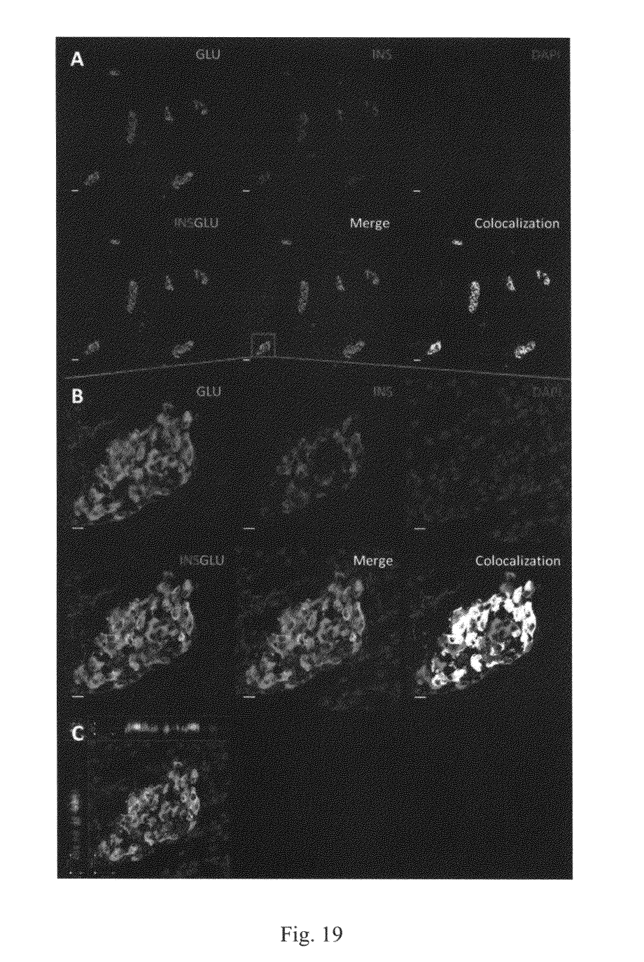

Image and Statistical Analysis—All images were acquired by confocal scanning microscopy (Radiance 2100/AGR-3Q BioRad Multi-photon Laser Point Scanning Confocal Microscope). Images were processed with Image J software. The colocalization patterns in FIG. 4, FIGS. 18 and 19 were determined by the colocalization threshold algorithm in WCIF image J. Confocal z stacks were captured at 0.5 lm increments. Three dimensional reconstructions and measurements were performed by using Volocity software (version 5.3). Statistical significance of cell-specific changes in controls versus experimental groups was calculated by an unpaired Student's t-test. We considered p-values below 0.05 as statistically significant. For all statistical analysis, Graphpad Prism 5 was used. All results are expressed as mean±SD.

### Embodiment 1: β-Cell Neogenesis Following PDL Plus Alloxan is Rapid and Robust

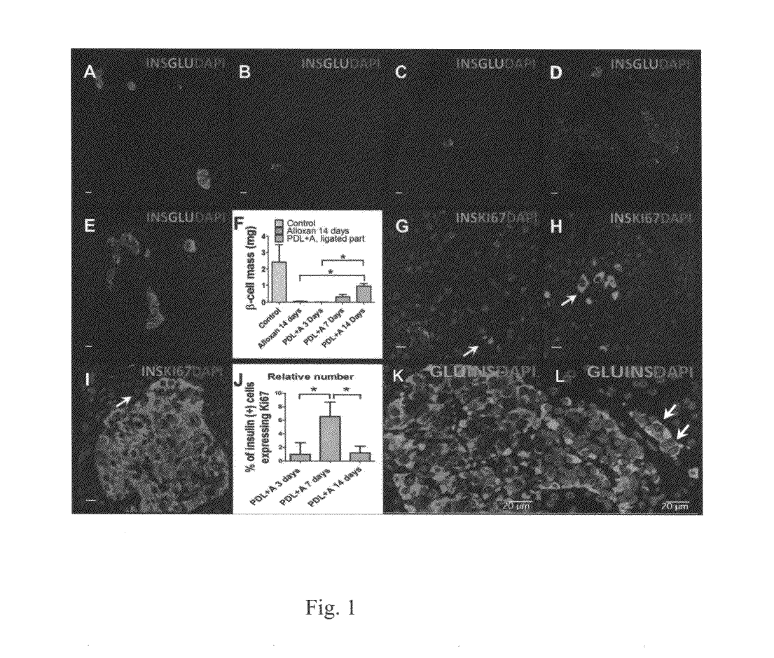

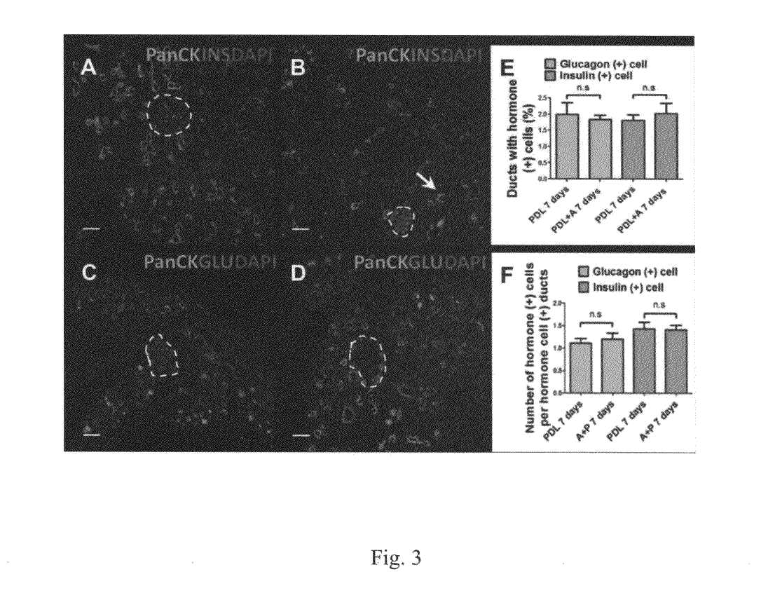

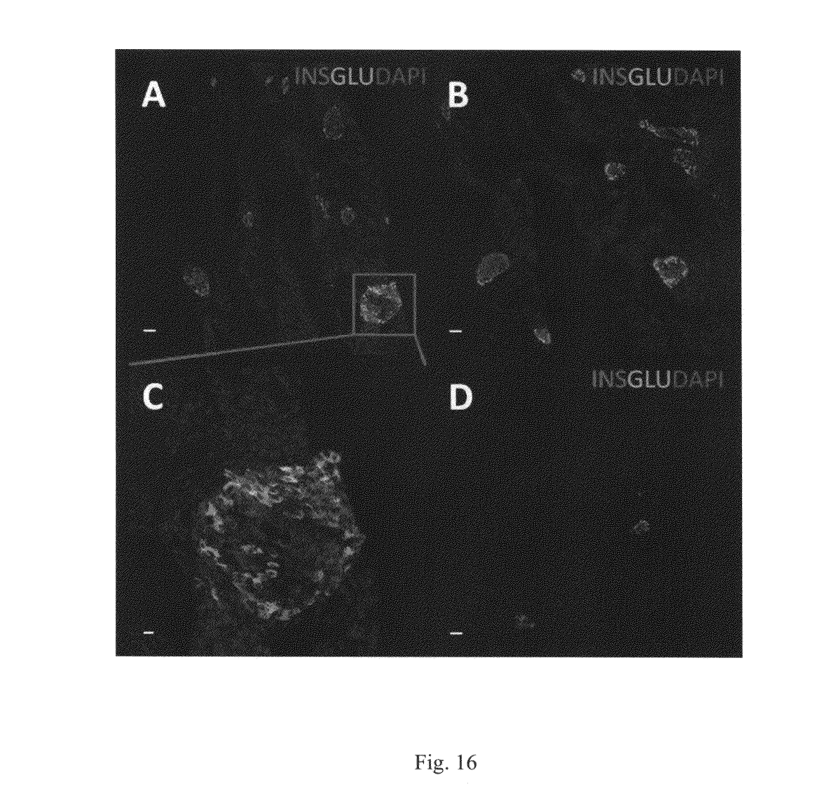

To determine the extent to which β-cell mass in adults can be regenerated by neogenesis, as opposed to replication, we combined PDL with elimination of pre-existing β-cells using the β-cell toxin alloxan. Following PDL plus alloxan, greater than 99% of β-cells were eliminated (compare FIG. 1A with FIG. 1B, quantitated in FIG. 1F), leaving residual clusters of α-cells marking the location of pre-existing islets (FIG. 1B). Two weeks later, there was no evidence of β-cell regeneration with alloxan treatment alone (FIG. 1C, FIG. 16D, quantitated in FIG. 1F). PDL alone resulted in the appearance of insulin- and glucagon positive cells within the hyperplastic ducts that are present in that model, but such cells were uncommon (FIG. 3B, 3D). PDL plus alloxan resulted in a dramatically different regenerative response than alloxan alone. Two weeks following PDL plus alloxan, large islets were found in the ligated but not unligated part of the pancreas (FIG. 1E, FIG. 16A-16C, quantitated in FIG. 1F). Some islets present 2 weeks after PDL plus alloxan were substantially larger than control islets (compare FIGS. 1A, 4B with FIG. 1E). Thus, the combination of alloxan and PDL resulted in the induction of rapid and robust β-cell regeneration. However, while the α-cells existed in a small cluster 3 days following alloxan treatment, by 14 days, the islets had the same relative arrangement of α- and β-cells as islets from control animals, with the α-cells surrounding the β-cells. This suggests that the sorting mechanism responsible for separating α- and β-cells during embryonic development was preserved in the adult pancreas.

With reference to FIG. 1 (K)˜(L). Representative pancreatic sections from an adult human patient without pancreatitis are shown in (K), and from an adult human patient with pancreatitis in (L). Endocrine cells in the adult human pancreas did not coexpress insulin (red) and glucagon (green) (K), but such double positive cells were found in patients with pancreatitis (L, indicated by arrows). Scale bar=20 um (K and L).

### Embodiment 2: β-Cells in the PDL Plus Alloxan Model are Newly Formed and Do Not Arise by Replication of Pre-Existing β-Cells

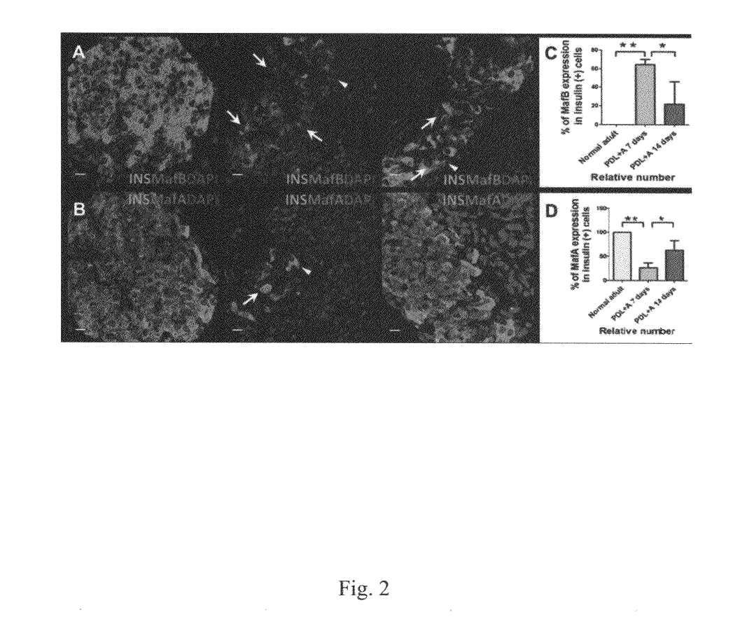

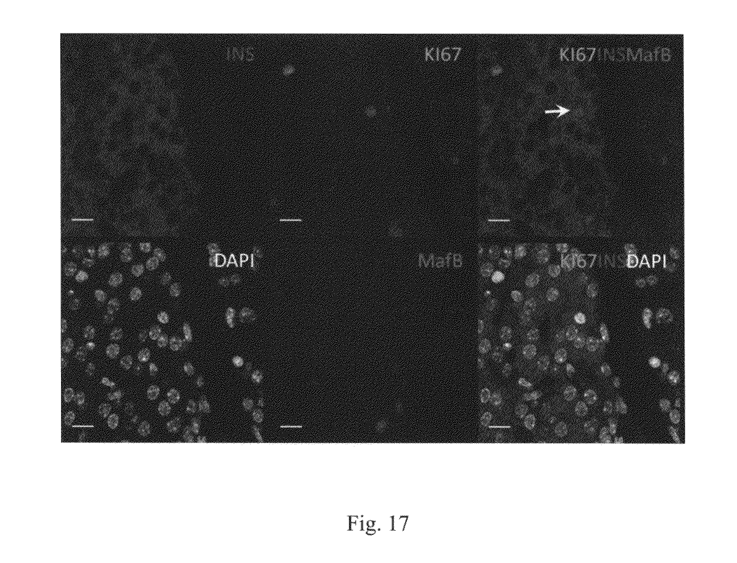

The β-cells that appeared at 2 weeks following PDL plus alloxan must have arisen either by replication of pre-existing β-cells that were not eliminated by alloxan or by neogenesis from a precursor. Given that almost all pre-existing β-cells were eliminated by alloxan, residual β-cells (less than 1% of pre-existing β-cells, FIG. 1) could account for regeneration only if there was highly efficient and rapid replication of the rare remaining β-cells. To address this possibility, we determined the frequency of β-cell replication at different times following PDL plus alloxan. By Ki67 immunohistochemistry, the frequency of replicating insulin-positive cells 3 days following alloxan plus PDL was very low (0.98%; FIG. 1G, quantified in FIG. 1J). It increased transiently to 6% by day 7 (FIG. 1H), but had decreased to basal levels by day 14 (FIG. 1I, quantified in FIG. 1J). Given the low level of β-cell replication, the small number of residual β-cells cannot account for the insulin-positive cells at days 7 and 14. To further examine the nature of the β-cells that were found at 7 days following PDL plus alloxan, we examined the expression of the transcription factors MafA and MafB. MafB is expressed in α-cells and in developing β-cells, and it is then replaced by MafA as β-cells mature. In the adult, all β-cells express MafA (FIG. 2B, 2D), with no MafB expression being detectable (FIG. 2A, 2C). When mature β-cells replicate, MafA continues to be expressed, with no dividing β-cells expressing MafB (FIG. 17). Thus, if the insulin-positive cells found following PDL plus alloxan were derived from pre-existing β-cells that survived the alloxan treatment, they should be uniformly positive for MafA, while if they arose from a neogenic process, they could express either MafA or MafB, depending on the extent of maturation.

Seven days following PDL plus alloxan, 64% of insulin positive cells expressed MafB (FIG. 2A, 2C), while 26% expressed MafA (FIG. 2B, 2D). This indicates that the great majority of insulin-positive cells must be newly formed and have arisen from a source other than replication of pre-existing β-cells, and further indicates that the neogenic process involves an intermediate that has at least some characteristics of an immature β-cell. By 14 days following alloxan plus PDL, the ratio of MafB/MafA expressing β-cells had reversed, with 63% expressing MafA (FIG. 2B, 2D), and only 22% expressing MafB (FIG. 2A, 2C), as would be expected with β-cell maturation.

### Embodiment 3: Neogenic β-Cells do not Arise Predominantly From Progenitors in the Duct

Having ruled out replication of pre-existing β-cells as a source of the regenerating β-cells following PDL plus alloxan, we concluded that the vast majority of insulin-positive cells must have arisen by neogenesis from a progenitor. Although substantial controversy exists, a number of studies have reported that duct epithelial cells can function as endocrine progenitors in the PDL model. Thus, it was important to examine the possibility that duct cells were the source of the neogenic β-cells following PDL plus alloxan. Given the tremendous increase in the number of β-cells that occurred following PDL plus alloxan compared with PDL alone, where none of the pre-existing β-cells were eliminated, the frequency of insulin-positive cells within ducts would have to be much higher in PDL plus alloxan compared with PDL alone if duct cells were the source of the neogenic β-cells. Thus, we examined the occurrence of insulin-positive cells within ducts in the PDL and PDL plus alloxan models, finding that there was no difference in the number of ducts that contained insulin-positive cells (FIG. 3A, 3B, 3E) or in the number of insulin-positive cells per duct (FIG. 3F). As it was recently shown that ectopic expression of Pax4 under the control of the glucagon promoter could shift the lineage of newly formed glucagon-positive cells in ducts to a β-cell lineage, we examined the occurrence of glucagon-positive cells within ducts. Similar to the insulin-positive cells, there was no difference in the number of ducts that contained glucagon-positive cells (FIG. 3C, 3D, 3E) or in the number of glucagon-positive cells per duct (FIG. 3F). Thus, we concluded that duct-associated insulin or glucagon-positive cells were highly unlikely to be the major source of neogenic β-cells. Additional evidence that the neogenic β-cells were not arising primarily from ducts came from the fact that the insulin-positive cells appeared in large numbers in association within pre-existing islets primarily composed of the α-cells that persisted following alloxan treatment (FIGS. 1D, 3A, 7A) rather than outside the pre-existing islets. This led us to conclude that the neogenic β-cells were likely to be arising from source located within the pre-existing islets.

### Embodiment 4: α-Cells Replicate and Differentiate Efficiently Into β-Cells in the PDL and Alloxan Model

Apart from β-cells, the two most common cell types in the islet are α- and d-cells. Because δ-cells have been reported to trans-differentiate into β-cells following low-dose streptozotocin treatment, we performed double-labeling by immunohistochemistry to look for transitional cells coexpressing somatostatin and insulin. A very low frequency of coexpressing cells was found (FIG. 18), making it unlikely that this pathway was a significant contributor to the large number of neogenic β-cells in the islet.

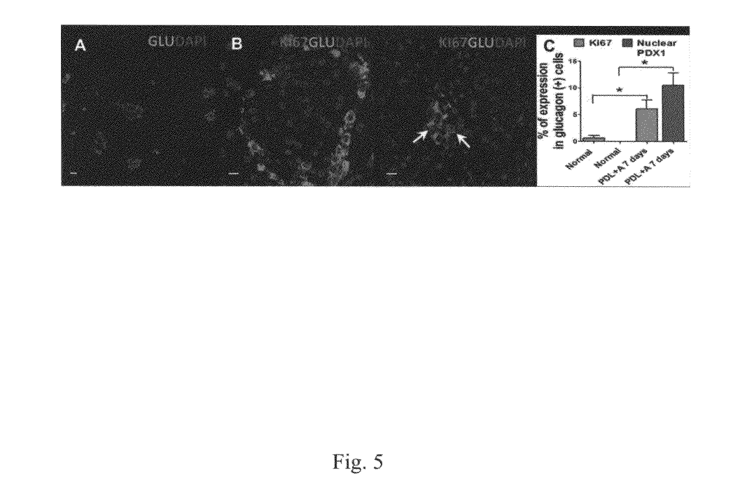

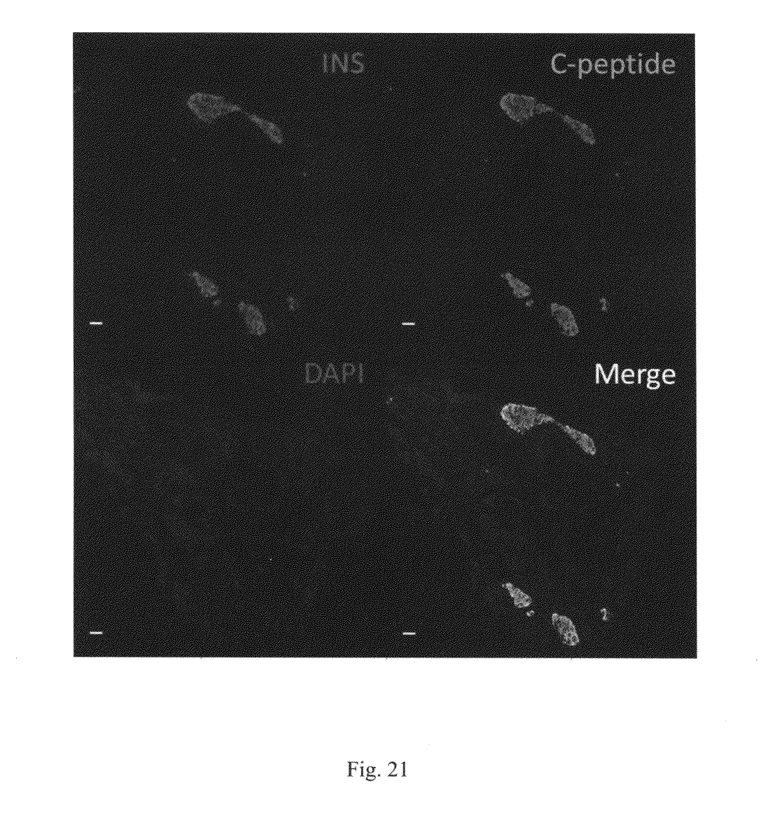

By 7 days following PDL plus alloxan, α-cell hyperplasia and numerous β-cells were present (FIGS. 1D, 5A). This was not found with alloxan (FIG. 1C) or PDL (FIG. 3D) alone. The replication rate of glucagon-positive cells increased from almost undetectable levels to 5% (FIG. 5B, 5C). Strikingly, at 1 week following PDL plus alloxan, 58% of insulin-positive cells coexpressed glucagon (FIG. 4A), suggesting that α-cells could be the source of the neogenic β-cells. By 14 days following PDL plus alloxan, colocalization had decreased. Overall 21% of insulin-positive cells coexpressed glucagon (FIG. 4D). However, in some cases, substantial colocalization of insulin and glucagon was still present at 14 days (FIG. 4B, 4C, FIG. 19). Insulin-positive cells at day 14 also expressed C-peptide (FIG. 21).

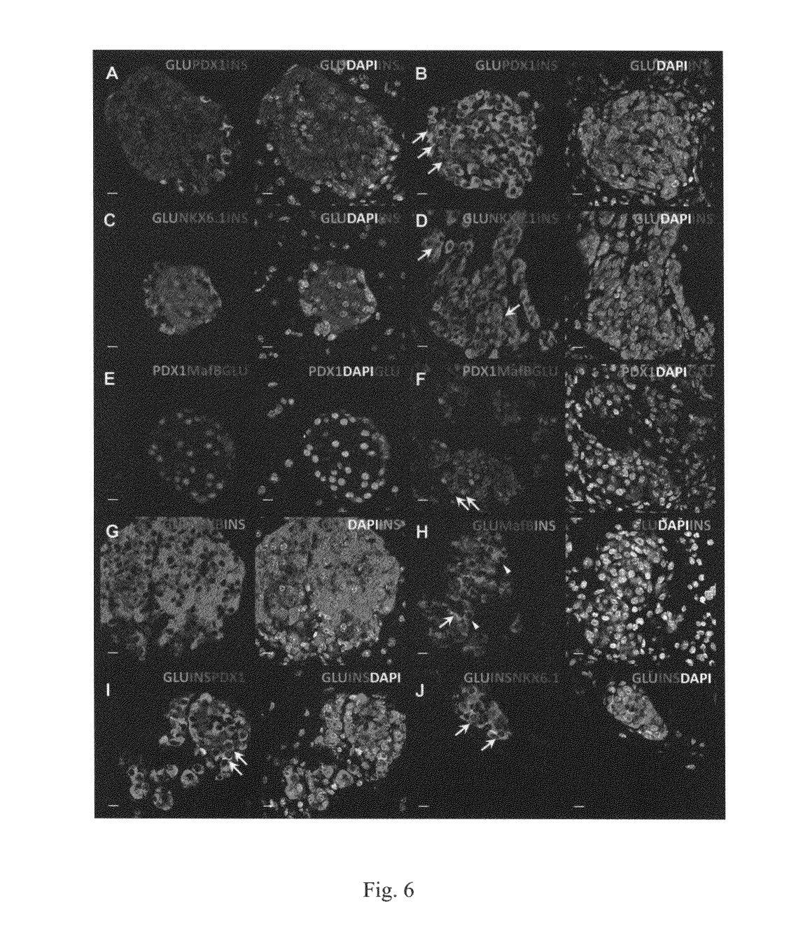

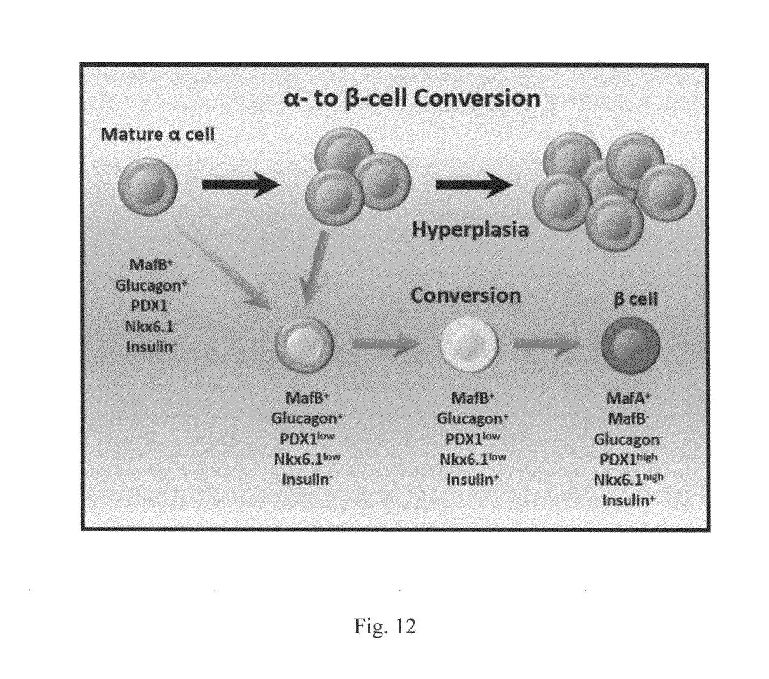

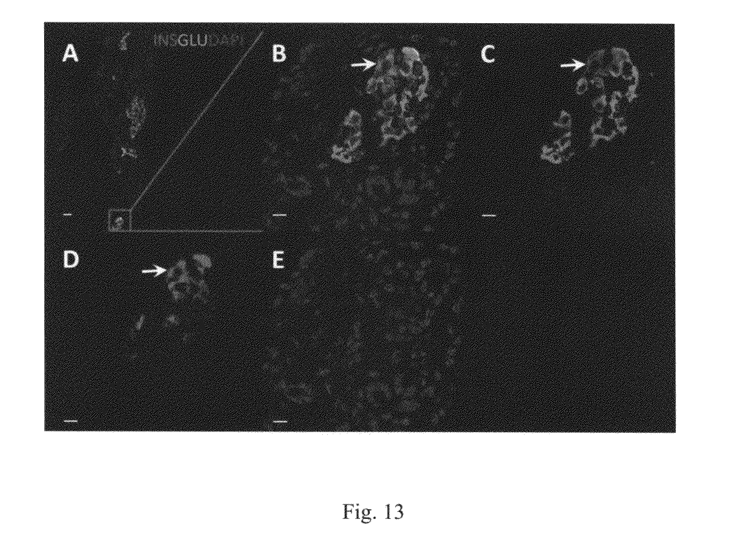

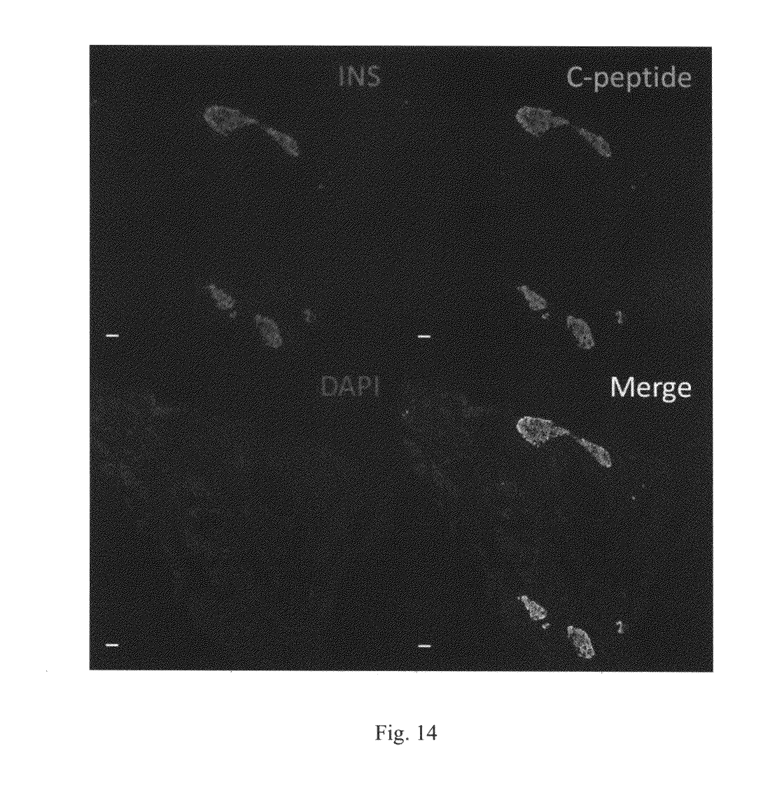

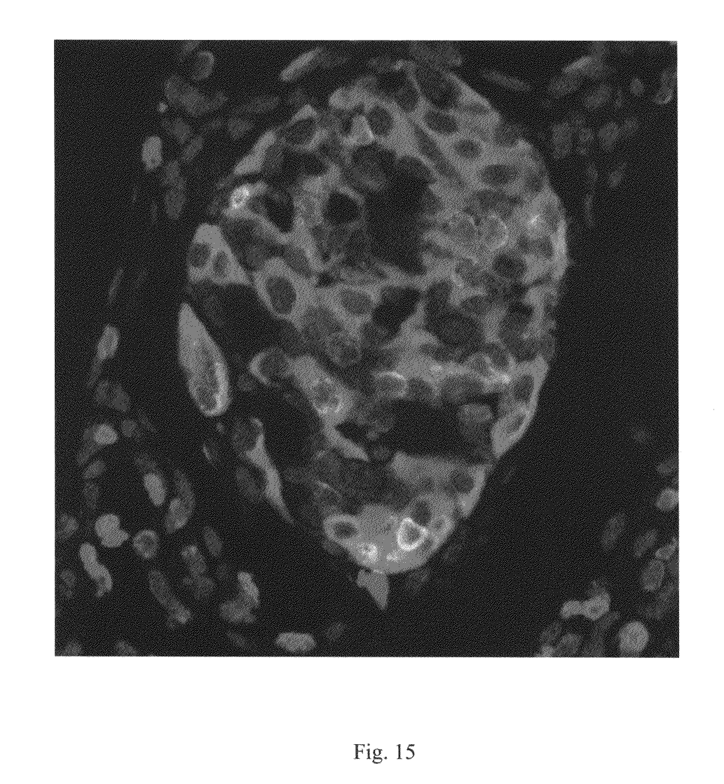

If α-cells were the source of neogenic β-cells, the transcription factors that contribute to cell type-specific hormone expression should play a role in α-cell to β-cell conversion, and intermediates expressing both β-cell and α-cell markers should be present. Thus, we determined the expression of the transcription factors MafA, PDX-1, and Nkx6.1, which in the adult pancreas are restricted to β-cells and MafB, which is restricted to mature α-cells. (FIGS. 2, 6A, 6C, 6E, 6G). In the PDL plus alloxan 11% of α-cells began to express PDX1 within 1 week (FIGS. 5C, 6B). Some of the α-cells expressing PDX-1 started to coexpress insulin (FIG. 6I). α-cells expressing Nkx6.1 (FIG. 6D), another β-cell marker, as well as insulin, and Nkx6.1 (FIG. 6J) were also found, suggesting that the induction of Nkx6.1 in α-cells was also an early event in α-cell to β-cell conversion. Intermediates coexpressing glucagon, PDX-1, MafB (FIG. 6F) and insulin, glucagon, MafB (FIG. 6H) were present as well. Compared with normal adult pancreas, the frequency of MafB expression in insulin-positive cells increased at day 7 and then decreased at day 14, suggesting that the expression of MafB is still preserve the early stage of α-cell to β-cell conversion, but is gradually turned off and replaced by MafA as neogenic β-cells become mature (FIG. 2). The model of α- to β-cell conversion is summarized in FIG. 12.

### Embodiment 5: α-Cell Replication and α- to β-Cell Conversion are Independent Processes

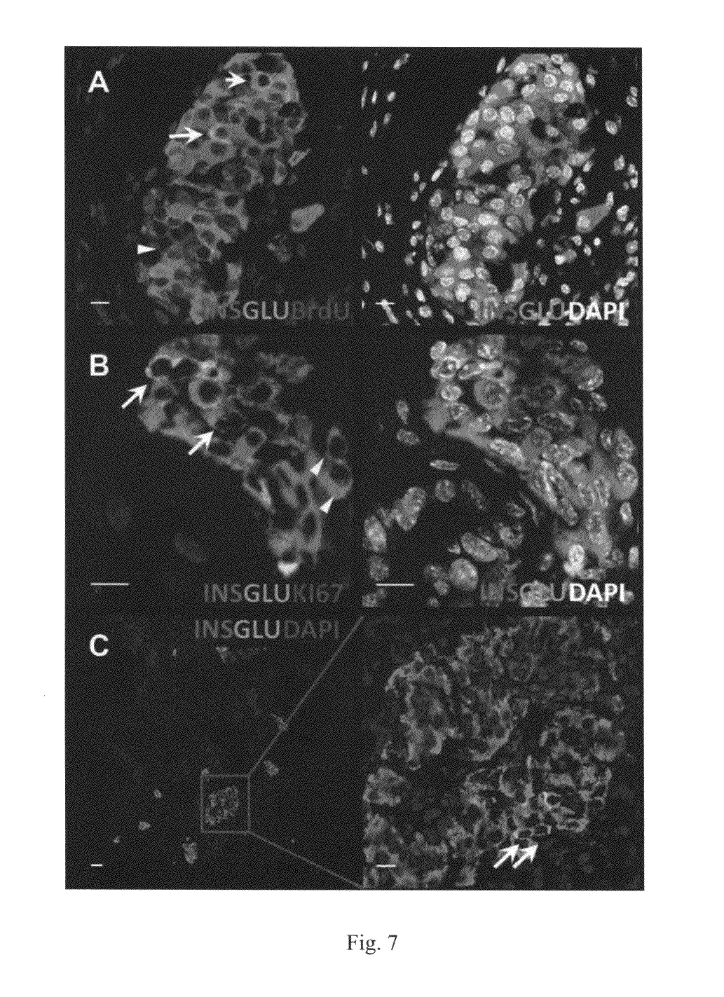

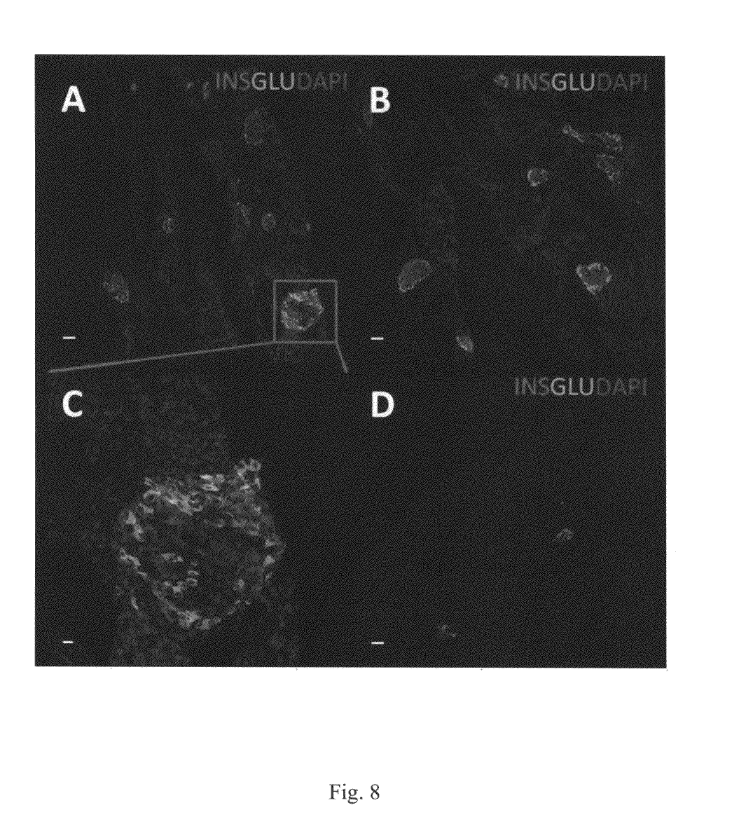

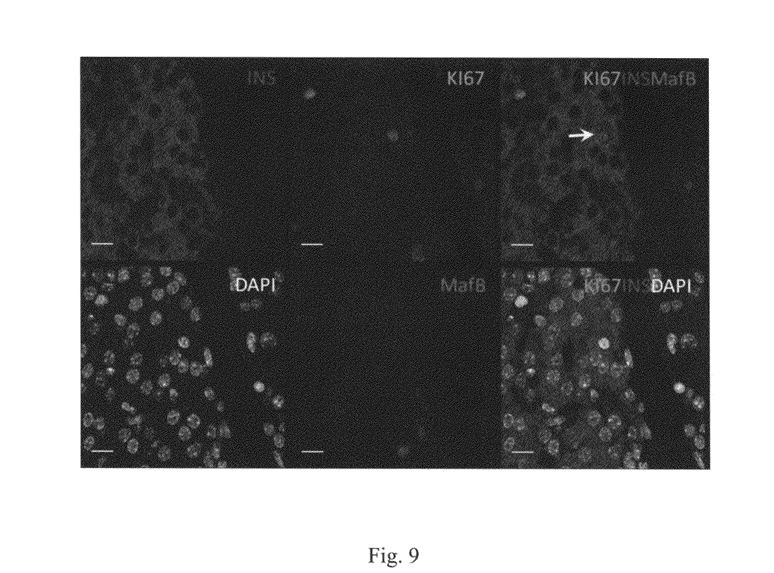

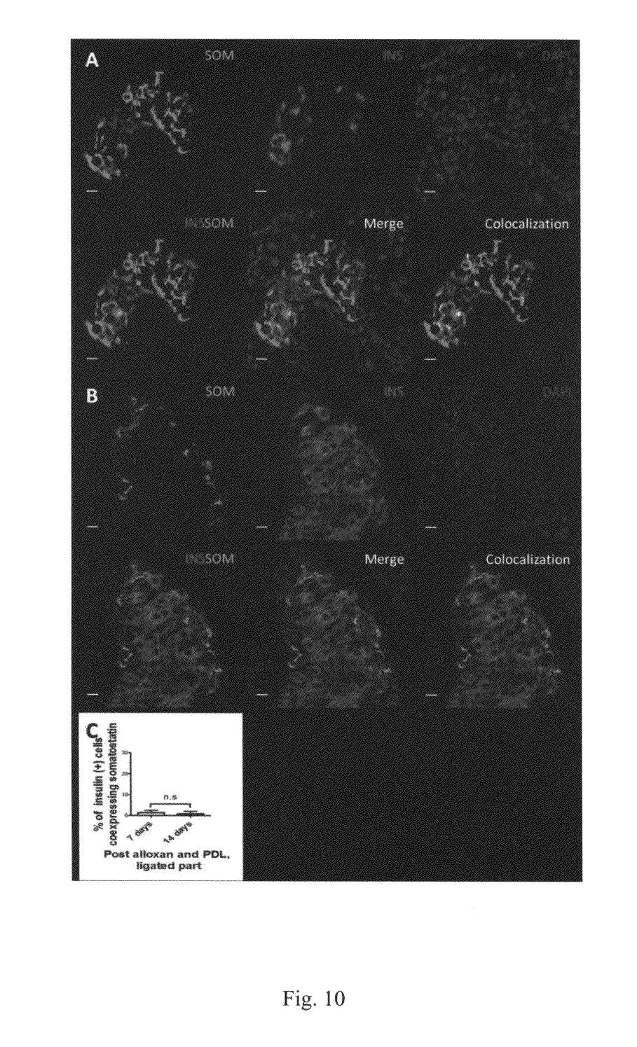

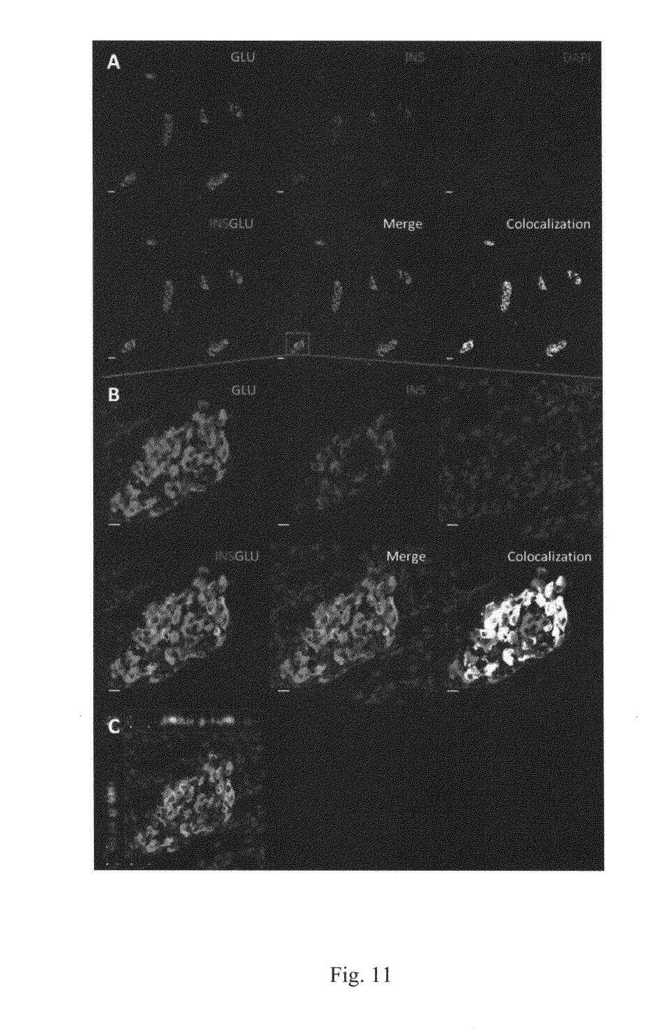

The occurrence of both α-cell hyperplasia and α- to β-cell conversion raised the possibility that the two phenomena might be linked, that is, that asymmetric division of replicating α-cells might be required for conversion to β-cells. The alternative was direct α- to β-cell conversion through a non-replicative intermediate. To distinguish between those possibilities, we performed continuous labeling with BrdU. If replication was required for β-cell neogenesis, then almost all β-cells present at 14 days following PDL plus alloxan should be positive for BrdU, while if replication and β-cell neogenesis from α-cells were unlinked, then neogenic β-cells could be either BrdU-positive or negative. Consistent with the latter model, we found insulin-positive cells that were BrdU-negative and BrdU-positive (FIG. 7A). Interestingly, we found numerous cells that were negative for BrdU but coexpressed insulin and glucagon, consistent with direct conversion without a replicative intermediate (FIG. 7A). This was further supported by examination of Ki67, which was absent from many cells coexpressing insulin and glucagon (FIG. 7B). Some α-cells remained negative for BrdU at 14 days following PDL plus alloxan (FIG. 7A), consistent with an origin of these α-cells from preexisting islets rather than neogenic α-cells from a replicative precursor, for example, a duct cell. Overall, it shows that α-cells could directly convert into β-cells with or without intervening replication and asymmetric division of α-cells is not required to generate new β-cells. The processes of α-cell replication and α- to β-cell conversion are unlinked in the PDL plus alloxan model.

### Embodiment 6: Effect of Age on β-Cell Neogenesis in the PDL Plus Alloxan Model

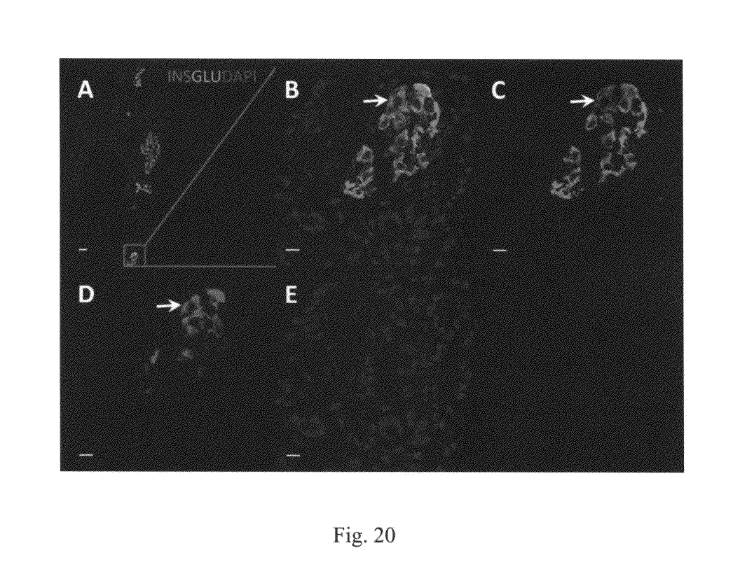

β-cell replication declines precipitously with age, but little is known about the effect of age on β-cell neogenesis, as most studies of β-cell regeneration have been performed in juvenile animals. Thus, we examined mature mice (7 months of age) for β-cell neogenesis in the PDL plus alloxan model. Although there was a decline in β-cell neogenesis with age, substantial numbers of neogenic β-cells still formed in 7-month-old mice (FIG. 7C, FIG. 20). This indicates that induction of β-cell neogenesis may be a viable approach for the treatment of diabetes even in mature adults.

The results presented here demonstrate for the first time that β-cell neogenesis from α-cells can be a rapid and robust process, resulting within 2 weeks in the formation of islets in which essentially all of the β-cells are neogenic (FIG. 4). Most previous studies using different damage models such as partial pancreatectomy concluded that β-cell neogenesis did not occur, at least to a significant extent. Even studies in which β-cell neogenesis was proven to occur, including applicant's previous study with human pancreatic exocrine cells, did not demonstrate a high efficiency of β-cell differentiation from progenitors, leading to skepticism about the extent to which β-cell neogenesis could occur and consequent doubts about the possibility of it being a viable route to enhance β-cell mass in patients with diabetes. A recent study in which essentially complete elimination of β-cells was accomplished using a transgenic approach to express the diphtheria toxin receptor in β-cells also found evidence for α- to β-cell conversion, although it took several months to generate significant number of β-cells. Also, the efficiency of α- to β-cell conversion in that model was much lower than with PDL plus alloxan. The rapidity and efficiency of in vivo α- to β-cell conversion in our model makes it much easier to study the process and also facilitates mechanistic studies required for future clinical translation.

The fact that the great majority of insulin-positive cells at day expressed MafB, together with the almost complete β-cell ablation and low β-cell replication rate, excluded the possibility that pre-existing β-cells were the origin of new β-cells. We scrutinized carefully the conversion process to define the nature of intermediate cells. The intermediate cells coexpressing multiple α-cell and β-cell markers, providing strong evidence for α-cell to β-cell conversion. The rapidity of the process in our model compared with β-cell ablation using diphtheria toxin allowed us to carefully examine the intermediates over time. Furthermore, the continuous BrdU labeling experiment and Ki67 expression showed that many transitional cells coexpressing insulin and glucagon during α-cell to β-cell conversion did not incorporate BrdU. This provided evidence that those insulin-positive cells were formed by direct conversion from adult α-cells without replication rather than from other potential progenitors, such as ductal cells or rare putative stem cells, which would need to proliferate extensively to generate the large number of β-cells present following PDL plus alloxan. Because of the large literature supporting a ductal origin for neogenic β-cells, we examined that possibility in great detail in our model, but there was no change in the frequency of endocrine neogenesis from ducts following PDL plus alloxan compared with PDL alone.

Some have put forth the possibility that β-cells might arise from sources, such as mesenchymal cells and neural cells within islets or dedifferentiated β-cells. However, such cells cannot account for the time course of hormone expression in our model, in which large numbers of cells expressing glucagon alone begin to coexpress insulin and then progress to mature β-cells.

Conventionally, differentiation of a stem/progenitor cell involves replication of the progenitor with asymmetric division to provide for self-renewal of the stem/progenitor population as well as the formation of progeny with a different differentiated state. However, there has been increasing interest in direct conversion of one cell type into another. For example, direct conversion of fibroblasts into neurons and acinar into endocrine cells have been reported. But, in those cases, conversion required the introduction of multiple genes into the precursor cell. The direct conversion of α- to β-cells is described here occurred under the influence of endogenous factors, raising the possibility hat this phenomenon occurs normally as part of tissue homeostasis or in response to damage stimuli.

The signals that control α-cell proliferation and conversion into β-cells remain unknown. α-cell proliferation occurs in a number of settings, including type 2 diabetes and selective deletion of the insulin receptor in α-cells. However, in most cases, α-cell proliferation does not appear to be accompanied by substantial β-cell neogenesis. In PDL plus alloxan model, we found α-cells could rapidly replicate and differentiate to β-cells. However, the processes of α-cells replication and conversion into β-cells are unlinked. Thus, conversion may require an additional signal that is present in the environment of the duct-ligated pancreas. Ectopic expression of Pax4 in duct cells that expressed glucagon following PDL promoted insulin expression in those cells that could be inhibited by injection of glucagon. However, in that case, the genetic alteration occurred during development and Pax4 expression in glucagon-positive cells in more mature animals did not result in β-cell conversion, suggesting that factors in addition to Pax4 are important in α-cell to β-cell conversion. Our result which greatly accelerated α-cell to β-cell conversion in vivo is important, as (in contrast to β-cells) α-cells are retained in type 1 diabetes, are increased in number in type 2 diabetes, and are co-transplanted with β-cells in islet transplantation.

Thus, α-cells are present in essentially all of the clinical setting which β-cell regeneration is desirable. However, clinical translation would require a method that does not require irreversible surgical intervention as with PDL. Although PDL plus alloxan promoted efficient β-cell neogenesis from α-cells, the mice did not achieve normoglycemia, most likely due to the profound and continuing inflammation and disruption of normal organ homeostasis that occur following PDL.

It was found that even older animals were able to generate a considerable number of β-cells by α-cell conversion, albeit not as efficiently as in juvenile animals. Overall, the studies presented here provide strong evidence that β-cell neogenesis from α-cells can be a robust and efficient process and with further mechanistic understanding might be harnessed as a therapeutic for patients with diabetes.

The present invention presents a new model for β-cell neogenesis in which the β-cells arise primarily from mature α-cells in this model, combining PDL and alloxan, adult α-cells rapidly replicate and convert efficiently into β-cells. However, replication with asymmetric division is not required for α-cell to β-cell conversion. The rapid and efficient β-cell neogenesis following PDL plus alloxan provides an attractive model to study the mechanism of α-cell to β-cell conversion which is important for eventual clinical translation.

In accordance with the above embodiments, the present invention provides methods of identifying adult pancreatic endocrine stem cells and methods of identifying the existence of differentiation processes from adult pancreatic endocrine stem cells toward pancreatic β cell fate. Diabetes is a disease with high prevalence rate and serious complications and is an enormous burden on health care systems and economies throughout the world. In diabetic patients, β cell deficit is a major pathological feature and plays a key role in disease progression. β cell regeneration to replace the cells lost in diabetes is a promising approach to develop anti-diabetes therapies. However, in the past, because the adult pancreatic endocrine stem cells were unknown, many approaches to β-cell regeneration have failed. Herein, we identified adult pancreatic endocrine stem cells. The identified cells are shown to possess unique properties of stem cells, including self-renewal and differentiation into pancreatic β-cells. In addition, the present invention demonstrates that the cells can rapidly generate a large amount of β-cells. The adult pancreatic endocrine stem cells have the phenotype of “glucagon (+)”, “glucagon (+) and PDX1 (−)”, “glucagon (+) and NKX6.1 (−)”, or “glucagon (+) and PDX1 (−) and NKX6.1 (−)”. The presence of “glucagon (+) cells”, “glucagon (+) and PDX1 (−) cells”, or “glucagon (+) and PDX1 (−) and NKX6.1 (−) cells” indicates the presence of adult pancreatic endocrine stem cells and allows the definition of adult pancreatic endocrine stem cell domains in the adult pancreas. The present invention provides a way that therapies for beta cell regeneration and anti-diabetes can be directed, both generally and specifically, toward adult pancreatic endocrine stem cells.

The present invention also provides a method to identify the existence of differentiation processes from adult pancreatic endocrine stem cells toward pancreatic β-cell fate. The method comprises: (a) providing a sample of pancreatic tissue or pancreatic cells; and (b) detecting glucagon, pancreatic and duodenal homeobox 1(PDX1), NK6 homebox 1(NKX6.1), insulin and v-maf musculoaponeurotic fibrosarcoma oncogene homolog B(MafB) expression of the sample; wherein the existence of cells having the phenotype of expressing at least two markers selected from the group consisting of glucagon, PDX1, NKX6.1, insulin and MafB, indicates the existence of differentiation processes from adult pancreatic endocrine stem cells toward pancreatic beta cell fate.

Preferably, the existence of cells having the phenotype of expressing glucagon and NKX6.1 or cells having the phenotype of expressing glucagon and PDX1 or cells having the phenotype of expressing glucagon, PDX1 and MafB or cells having the phenotype of expressing insulin, glucagon and MafB or cells having the phenotype of expressing insulin, glucagon and PDX1 or cells having the phenotype of expressing insulin, glucagon and NKX6.1 or cells having the phenotype of expressing insulin and glucagon, indicates the existence of differentiation processes from adult pancreatic endocrine stem cells toward pancreatic beta cell fate.

The present invention also provides a method for generating new pancreatic beta cells from adult pancreatic endocrine stem cells which are pancreatic alpha cells in vivo in animals. The method comprises: (a) eliminating pre-existing pancreatic beta cells; and (b) blocking the flow of pancreatic juice or inducing apoptosis of acinar cells. Preferably, the elimination of pre-existing pancreatic beta cells in step(a) is by administration of alloxan or any methods to eliminate pre-existing pancreatic beta cells, and blocking of the flow of pancreatic juice in step(a) is by performing a pancreatic ductal ligation or methods to induce apoptosis of pancreatic acinar cells.

The present invention also provides a new animal model to generate new pancreatic beta cells from adult pancreatic endocrine stem cells which are alpha cells in vivo in animals. This new animal model is to perform pancreatic ductal ligation (PDL) and alloxan administration on animals. This new animal model can be utilized in screening and development of new factors or molecules or compounds or medicines or therapy protocols for diabetes. This new animal model also can be used for testing beta cell regeneration therapy and anti-diabetes therapy.

All of the references cited herein are incorporated by reference in their entirety.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of the present invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

What is claimed is:

1. A method for characterizing adult pancreatic endocrine stem cells, comprising:
(a) providing a sample of pancreatic tissue or pancreatic cells;
(b) detecting glucagon, pancreatic and duodenal homeobox 1(PDX1) and NK6 homebox 1(NKX6.1) expression of the sample; and
(c) quantitating the number of glucagon (+) cells of the sample; wherein the presence of glucagon (+) cells is indicative of the presence of adult pancreatic endocrine stem cells.

2. The method as claimed in claim 1 further comprising quantitating the number of glucagon (+) and PDX1 (−) and NKX6.1 (−) cells of the sample; wherein the presence of glucagon (+) and PDX1 (−) and NKX6.1 (−) cells is indicative of the presence of adult pancreatic endocrine stem cells.

3. The method as claimed in claim 1 further comprising quantitating the number of glucagon (+) and PDX1 (−) cells of the sample; wherein the presence of glucagon (+) and PDX1 (−) cells is indicative of the presence of adult pancreatic endocrine stem cells.

4. The method as claimed in claim 1 further comprising quantitating the number of glucagon (+) and NKX6.1 (−) cells of the sample; wherein the presence of glucagon (+) and NKX6.1 (−) cells is indicative of the presence of adult pancreatic endocrine stem cells.

5. The method as claimed in claim 1, wherein detection of glucagon, PDX1 and NKX6.1 expression is in individual promoter activity or RNA level or protein level.

6. A method for identifying the existence of differentiation processes from adult pancreatic endocrine stem cells toward pancreatic beta cell fate, comprising:
(a) providing a sample of pancreatic tissue or pancreatic cells; and
(b) detecting glucagon, pancreatic and duodenal homeobox 1(PDX1), NK6 homebox 1(NKX6.1), insulin and v-maf musculoaponeurotic fibrosarcoma oncogene homolog B(MafB) expression of the sample; wherein the existence of cells having the phenotype of expressing at least two markers selected from the group consisting of glucagon, PDX1, NKX6.1, insulin and MafB, indicates the existence of differentiation processes from adult pancreatic endocrine stem cells toward pancreatic beta cell fate.

7. The method as claimed in claim 6, wherein the existence of cells preferably having the phenotype of expressing glucagon and NKX6.1 indicates the existence of differentiation processes from adult pancreatic endocrine stem cells toward pancreatic beta cell fate.

8. The method as claimed in claim 6, wherein the existence of cells preferably having the phenotype of expressing glucagon, PDX1 and MafB indicates the existence of differentiation processes from adult pancreatic endocrine stem cells toward pancreatic beta cell fate.

9. The method as claimed in claim 6, wherein the existence of cells preferably having the phenotype of expressing insulin, glucagon and MafB indicates the existence of differentiation processes from adult pancreatic endocrine stem cells toward pancreatic beta cell fate.

10. The method as claimed in claim 6, wherein the existence of cells preferably having the phenotype of expressing insulin, glucagon and PDX1 indicates the existence of differentiation processes from adult pancreatic endocrine stem cells toward pancreatic beta cell fate.

11. The method as claimed in claim 6, wherein the existence of cells preferably having the phenotype of expressing insulin, glucagon and NKX6.1 indicates the existence of differentiation processes from adult pancreatic endocrine stem cells toward pancreatic beta cell fate.

12. The method as claimed in claim 6, wherein the existence of cells preferably having the phenotype of expressing insulin and glucagon indicates the existence of differentiation processes from adult pancreatic endocrine stem cells toward pancreatic beta cell fate.

13. The method as claimed in claim 6, wherein the existence of cells preferably having the phenotype of expressing glucagon and PDX1 indicates the existence of differentiation processes from adult pancreatic endocrine stem cells toward pancreatic beta cell fate.

14. The method as claimed in claim 6, wherein the detection of glucagon, PDX1, NKX6.1, insulin and MafB expression is in individual promoter activity or RNA level or protein level.

15. A method for generating new pancreatic beta cells from adult pancreatic endocrine stem cells which are pancreatic alpha cells in vivo in animals, comprising:
(a) eliminating pre-existing pancreatic beta cells; and
(b) inducing apoptosis of acinar cells.

16. The method as claimed in claim 15, wherein elimination of pre-existing pancreatic beta cells in step (a) is by administration of alloxan.

17. The method as claimed in claim 15, wherein blocking of the flow of pancreatic juice in step (a) is by performing a pancreatic ductal ligation.

18. The method as claimed in claim 15 being used as a model for identification of new therapeutic targets for beta cell regeneration.

19. The method as claimed in claim 15 being used as an anti-diabetes therapy.
